(19) United States
(12) Patent Application Publication
(10) Pub. No.: US 2025/0277241 A1
(43) Pub. Date: Sep. 4, 2025

(54) BIOFUEL PRODUCTION METHOD

(71) Applicant: TOYOTA JIDOSHA KABUSHIKI KAISHA, Toyota-shi (JP)

(72) Inventor: Tadayoshi SUMIYA, Toyota-shi (JP)

(73) Assignee: TOYOTA JIDOSHA KABUSHIKI KAISHA, Toyota-shi (JP)

(21) Appl. No.: 19/018,313

(22) Filed: Jan. 13, 2025

(30) Foreign Application Priority Data
Mar. 1, 2024 (JP) 2024-031268

(51) Int. Cl. C12P 7/10; C10L 8/00

(52) U.S. Cl. CPC C12P 7/10; C10L 8/00; C10L 2200/0469

(57) ABSTRACT

A biofuel production method that does not require a pH adjustment of a biomass raw material as a pretreatment is provided. A biofuel production method according to the present disclosure includes mixing a terrestrial-derived raw material, i.e., a terrestrial biomass, with a marine-derived raw material, i.e., a marine biomass, and performing fermentation using the mixed raw material, thereby producing ethanol.

### CROSS REFERENCE TO RELATED APPLICATIONS

This application is based upon and claims the benefit of priority from Japanese patent application No. 2024-031268, filed on Mar. 1, 2024, the disclosure of which is incorporated herein in its entirety by reference.

### BACKGROUND

The present disclosure relates to a biofuel production method.

When a biomass fuel is produced, it is required to increase the productivity of a target product such as the yield and the rate thereof in accordance with the characteristics of the biomass to be used.

For example, Patent Literature 1 discloses a technique for improving the productivity such as multiple parallel fermentation in which a saccharification step is adjusted to an optimum state using lignocellulose which is a terrestrial biomass.

Patent Literature 1: Japanese Unexamined Patent Application Publication No. 2020-039295

### SUMMARY

The inventors have found the following problem regarding a biofuel production method.

In the technology disclosed in Patent Literature 1, a pH adjustment of a biomass raw material is performed as a pretreatment depending on the biomass raw material. However, in order to improve the Life Cycle Assessment (LCA), it has been required to develop a biofuel production method that does not require a pH adjustment of a biomass raw material as a pretreatment.

The present disclosure has been made in view of the above-described problem and an object thereof is to provide a biofuel production method that does not require a pH adjustment of a biomass raw material as a pretreatment.

In order to achieve the above-described object, one aspect according to the present disclosure is a biofuel production method including:
mixing a terrestrial-derived raw material with a marine-derived raw material; and
performing fermentation using the mixed raw material, thereby producing ethanol.

According to the present disclosure, it is possible to provide a biofuel production method that does not require a pH adjustment of a biomass raw material as a pretreatment.

The above and other objects, features and advantages of the present disclosure will become more fully understood from the detailed description given hereinbelow and the accompanying drawings.

### BRIEF DESCRIPTION OF DRAWINGS

FIG. 1 is an enlarged schematic diagram of an example of a mixed biomass according to an embodiment;

FIG. 2 is a flowchart showing an example of a biofuel production method according to an embodiment; and

FIG. 3 is a graph showing an example of a surface potential and a particle size of biomass.

### DESCRIPTION OF EMBODIMENTS

Embodiments of the present disclosure will be described hereinafter in detail with reference to the drawings. The same or corresponding elements are denoted by the same reference symbols throughout the drawings, and redundant descriptions thereof will be omitted as necessary for the clarification of the description. Further, for facilitating the understanding of the present disclosure, the scale of each element in the drawings may differ from the actual scale thereof.

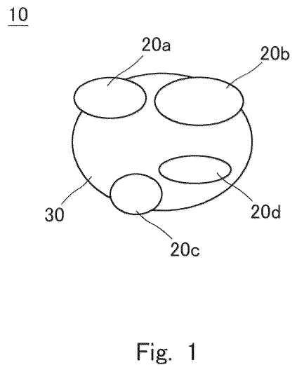

First, an example of a structure of a mixed biomass used in a biofuel production method according to this embodiment, that is, a mixed biomass according to this embodiment will be described with reference to FIG. 1. A mixed biomass 10 according to this embodiment is a mixture produced using a plurality of biomass raw materials, and is suitably used for ethanol brewing. As shown in FIG. 1, the mixed biomass includes a marine biomass 20 (20a, 20b, 20c, 20d) and a terrestrial biomass 30.

The marine biomass 20 is a marine-derived raw material. The marine biomass 20 is produced using, for example, seaweed. The terrestrial biomass 30 is a terrestrial-derived raw material. The terrestrial biomass 30 is produced using, for example, bamboo or sugarcane. The marine biomass 20 and the terrestrial biomass 30 have particle sizes different from each other. A method for measuring particle sizes of the terrestrial biomass 30 and the marine biomass 20 is not limited to a particular method. Particle sizes of the terrestrial biomass 30 and the marine biomass 20 may be measured as, for example, median diameters. For example, a particle size median diameter of the marine biomass 20 may be about ¼ of that of the terrestrial biomass 30.

FIG. 1 illustrates the case where the marine biomass 20 has a particle size smaller than that of the terrestrial biomass 30. However, the marine biomass 20 may have a particle size larger than that of the terrestrial biomass 30. In this case, since the marine biomass 20 and the terrestrial biomass 30 have particle sizes different from each other, the marine biomass 20 and the terrestrial biomass 30 are more homogeneously mixed and compounded than in the case where the marine biomass 20 and the terrestrial biomass 30 have substantially the same particle size.

The marine biomass 20 and the terrestrial biomass 30 usually have pHs different from each other since they are composed of different materials. Therefore, a pH of the mixed biomass 10 can be adjusted by adjusting a ratio of the mixture of the marine biomass 20 to that of the terrestrial biomass 30. Further, since the marine biomass 20 and the terrestrial biomass 30 are compounded in the mixed biomass 10, there is no unevenness in the pH inside the mixed biomass 10. Therefore, the pH of the whole mixed biomass 10 has been adjusted to an intended level suitable for ethanol fermentation. A ratio of the mixture of the marine biomass 20 to that of the terrestrial biomass 30 is determined so that the pH of the mixed biomass 10 becomes, for example, about 5 to 8.

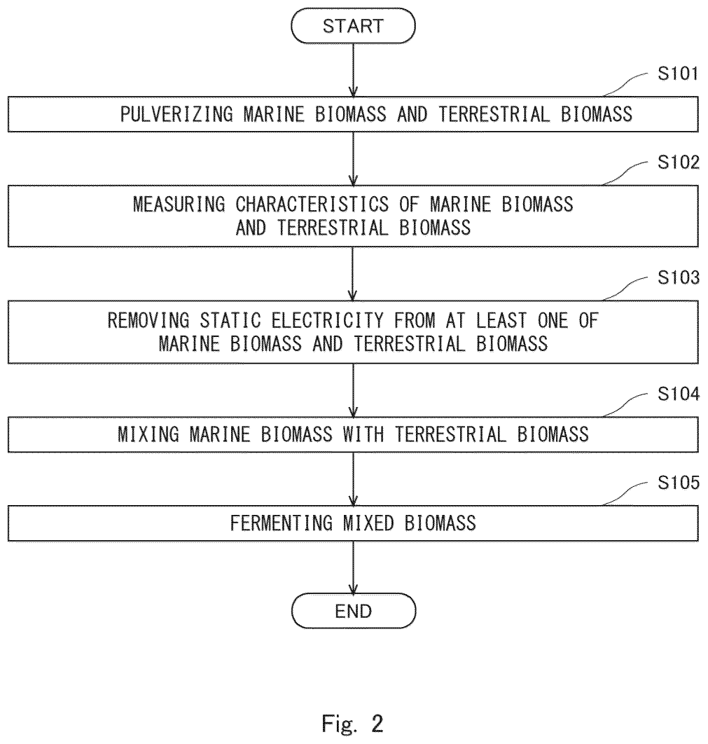

Next, a flow of the biofuel production method according to this embodiment will be described with reference to FIG. 2. In the biofuel production method according to this embodiment, first, the marine biomass 20 and the terrestrial biomass 30 are pulverized (Step S101). In Step S101, the marine biomass 20 and the terrestrial biomass 30 are pulverized so that they both have predetermined particle sizes, that is, particle sizes different from each other. Specifically, for example, the marine biomass 20 may be pulverized so that the particle size median diameter thereof becomes greater than or equal to 100 μm but smaller than or equal to 300 μm. Further, the terrestrial biomass 30 may be pulverized so that the particle size median diameter thereof becomes greater than or equal to 300 μm.

Next, characteristics of the marine biomass 20 and the terrestrial biomass 30 are measured (Step S102). In Step S102, specifically, a pH of the marine biomass 20 and a pH of the terrestrial biomass 30 are measured. A ratio of the mixture of the marine biomass 20 to that of the terrestrial biomass 30 and the like are determined based on a result of the measurement of the pH in Step S102. In Step S102, an electric charge of each of the marine biomass 20 and the terrestrial biomass 30 is preferably measured. It may be determined whether or not the static electricity needs to be removed from the marine biomass 20 and the terrestrial biomass 30 based on a result of the measurement of the electric charge in Step S102.

Next, the static electricity may be removed from at least one of the marine biomass 20 and the terrestrial biomass 30 (Step S103). Step S103 may be performed when it is determined in Step S102 that the static electricity needs to be removed. Note that, in the example shown in FIG. 1, although Step S103 is performed after Step S102, Step S103 may be performed in parallel with Step S102 or before Step S102. The means for removing the static electricity is not limited to particular means; for example, the static electricity may be removed by electrostatic adsorption using an ionizer.

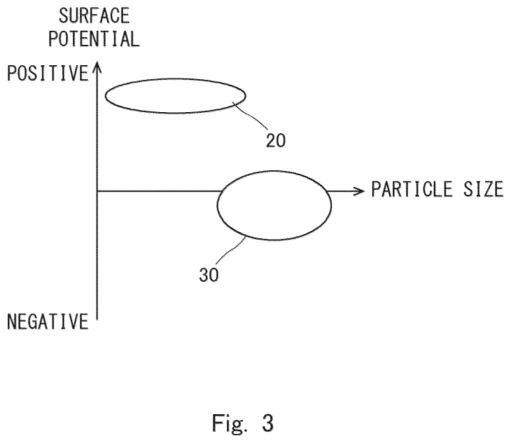

FIG. 3 shows a relationship between a surface potential and a particle size of each of the marine biomass 20 and the terrestrial biomass 30 when the static electricity is removed only from the terrestrial biomass 30. In the example shown in FIG. 3, the terrestrial biomass 30 has a particle size larger than that of the marine biomass 20. Therefore, in the example shown in FIG. 3, in order to reduce the amount of energy required to remove the static electricity, the static electricity is removed only from the terrestrial biomass 30. The terrestrial biomass 30 is normally positively charged. Therefore, the terrestrial biomass 30 is electrically brought close to neutral by removing the static electricity therefrom using an ionizer. Further, the marine biomass 20 is normally positively charged.

By performing Step S103, the marine biomass 20 and the terrestrial biomass 30 can be more homogeneously mixed and compounded by charging due to a potential difference between the marine biomass 20 and the terrestrial biomass 30.

Note that, in the example shown in FIG. 3, although the static electricity is removed only from the terrestrial biomass 30, the static electricity may be removed only from the marine biomass 20, or the static electricity may be removed from both the marine biomass 20 and the terrestrial biomass 30. Further, the terrestrial biomass 30 may be negatively charged and the marine biomass 20 may be positively and uniformly charged in order to further strengthen the composition of particles. In this case, electrostatic coupling between the terrestrial biomass 30 and the marine biomass 20 becomes stronger.

Referring back to FIG. 2, the description will be continued. Next, the marine biomass 20 and the terrestrial biomass 30 are mixed (Step S104). In Step S104, the marine biomass 20 and the terrestrial biomass 30 are mixed at the ratio of the mixture of the marine biomass 20 to that of the terrestrial biomass 30 determined based on the pHs measured in Step S102, to thereby obtain the mixed biomass 10. The marine biomass 20 and the terrestrial biomass 30 have particle sizes different from each other. Thus, in the mixed biomass 10, the marine biomass 20 and the terrestrial biomass 30 are homogeneously mixed and compounded. Therefore, there is no unevenness in the pH inside the mixed biomass 10.

In general, biomass contains various components, and these components are often localized. Charging characteristics change depending on the content ratio and localization of the components. Therefore, simply mixing different kinds of biomass causes particles to come into contact with each other and have a positive electric effect on each other, with the result that, for example, the particle are often aggregated. Therefore, in order to reliably compound different kinds of biomass, it is preferable to adjust a charged state of each biomass before they are mixed.

Next, Ethanol is produced by performing fermentation using the mixed biomass 10 (Step S105). In Step S105, in normal times, after a saccharification step by an enzymatic reaction is performed, a fermentation step using yeast is performed. Regarding a detailed procedure for carrying out Step S105, a known method may be employed. In the biofuel production method according to this embodiment, a pH of the mixed biomass is adjusted by mixing the marine biomass 20 with the terrestrial biomass 30. Therefore, the mixed biomass having a pH suitable for saccharification and fermentation steps can be prepared without adding alkali or acid thereto. As described above, the biofuel production method according to this embodiment does not require a pH adjustment of a biomass raw material as a pretreatment. Therefore, the LCA can be improved and the production cost can be reduced.

Further, in the biofuel production method according to this embodiment, since the marine biomass 20 is compounded with the terrestrial biomass 30, reactions in saccharification and fermentation steps proceed in a state in which a Ph of the whole biomass is maintained at a predetermined level. Therefore, reaction inhibition caused by local alkali or local acid does not occur in any of the steps of the biofuel production method, and hence ethanol can be efficiently produced.

Note that the present disclosure is not limited to the above-described embodiments and may be changed as appropriate without departing from the scope and spirit of the present disclosure.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A biofuel production method comprising:
mixing a terrestrial-derived raw material with a marine-derived raw material; and
performing fermentation using the mixed raw material, thereby producing ethanol.

2. The biofuel production method according to claim 1, wherein
a particle size median diameter of the terrestrial-derived raw material is greater than or equal to 300 μm, and
a particle size median diameter of the marine-derived raw material is greater than or equal to 100 μm but smaller than or equal to 300 μm.

3. The biofuel production method according to claim 1, further comprising removing a static electricity of at least one of the terrestrial-derived raw material and the marine-derived raw material prior to the mixing.
